(19) United States
(12) Patent Application Publication
(10) Pub. No.: US 2022/0253747 A1
(43) Pub. Date: Aug. 11, 2022

(54) Likelihood Ratios for Out-of-Distribution Detection

(71) Applicant: Google LLC, Mountain View, CA (US)

(72) Inventors: Jie Ren, Mountain View, CA (US); Balaji Lakshminarayanan, Mountain View, CA (US); Peter Junteng Liu, Mountain View, CA (US); Joshua Vincent Dillon, Mountain View, CA (US); Roland Jasper Snoek, Mountain View, CA (US); Ryan Poplin, Mountain View, CA (US); Mark Andrew DePristo, Mountain View, CA (US); Emily Amanda Fertig, Mountain View, CA (US)

(21) Appl. No.: 17/616,494

(22) PCT Filed: May 26, 2020
(86) PCT No.: PCT/US2020/034475

§ 371 Date: Dec. 3, 2021

Related U.S. Application Data
(60) Provisional application No. 62/857,774, filed on Jun. 5, 2019.

(51) Int. Cl. G06N 20/00; G06N 3/12

(52) U.S. Cl. CPC G06N 20/00; G06N 3/123

(57) ABSTRACT

The present disclosure is directed to systems and method to perform improved detection of out-of-distribution (OOD) inputs. In particular, current deep generative model-based approaches for OOD detection are significantly negatively affected by and struggle to distinguish population level background statistics from semantic content relevant to the in-distribution examples. In fact, such approaches have even been experimentally observed to assign higher likelihood to OOD inputs, which is opposite to the desired behavior. To resolve this problem, the present disclosure proposes a likelihood ratio method for deep generative models which effectively corrects for these confounding background statistics.

### RELATED APPLICATIONS

This application claims priority to and the benefit of U.S. Provisional Patent Application No. 62/857,774, filed Jun. 5, 2019. U.S. Provisional Patent Application No. 62/857,774 is hereby incorporated by reference in its entirety.

### FIELD

The present disclosure relates generally to machine learning. More particularly, the present disclosure relates to systems and method for improved detection of out-of-distribution inputs. The term “in-distribution” is used to describe a dataset which is a sample from a certain data distribution. The distribution may be associated with a plurality of “in-distribution classes”, which describe respective portions of the space of possible in-distribution datasets. By contrast, the term “out-of-distribution” (ODD) refers to a dataset which is not a sample from the distribution. The distribution may for example be a distribution of training examples which have been used to train a machine learning system. The datasets may be datasets obtained by observations and/or measurements of the real world, especially physical, biological, medical or chemical objects or events in the real world. For example, they may be datasets encoding nucleic acid sequences observed in the real-world, and/or datasets encoding sensor data (e.g. images or sound) captured by one or more sensors (e.g. cameras including video cameras, and/or microphones).

### BACKGROUND

For many machine learning systems, being able to detect data that is anomalous or significantly different from that used in training can be critical to maintaining safe and reliable predictions. This is particularly important for deep neural network classifiers which have been shown to incorrectly classify OOD inputs into in-distribution classes with high confidence. This behavior can have serious consequences when the predictions inform real-world decisions such as medical diagnosis, e.g., falsely classifying a healthy sample as pathogenic or vice versa can have extremely high cost. As such, the importance of dealing with OOD inputs, also referred to as distributional shift, has been recognized as an important problem for AI safety.

One example sub-problem in which OOD detection is important is that of bacterial identification and many other types of medical diagnosis. For example, diagnosis and treatment of infectious diseases, such as sepsis, relies on the accurate detection of bacterial infections in blood. Several machine learning methods have been developed to perform bacteria identification by classifying known genomic sequences, including deep learning methods which are state-of-the-art.

However, even if neural network classifiers achieve high accuracy as measured through cross-validation, deploying them is challenging as real data is highly likely to contain genomes from unseen classes not present in the training data. In particular, different bacterial classes continue to be discovered gradually over the years and it is estimated that 60%-80% of genomic sequences belong to as yet unknown bacteria. Thus, training a classifier on existing bacterial classes and deploying it may result in OOD inputs being wrongly classified as one of the classes from the training data with high confidence. In addition, OOD inputs can also be the contaminations from the bacteria's host genomes such as human, plant, fungi, etc., which also need to be detected and excluded from predictions. Thus having a method for accurately detecting OOD inputs is critical to enable the practical application of machine learning methods to this important problem. In addition to this example sub-problem, the above-described dynamic and challenge is generalizable to many different scenarios/problems, including any scenario in which OOD inputs may be present and mis-classification of such OOD inputs would be problematic.

In the current state of the art, one popular strategy for detecting OOD inputs is to train a generative model on training data and use that to detect OOD inputs at test time. However, recent research has shown that deep generative models trained on image datasets can assign higher likelihood to OOD inputs (that is, the deep generative models can erroneously classify the OOD input as being in one of the in-distribution classes with higher likelihood than the deep generative models assign to an in-distribution input). Thus, these existing approaches may, in some scenarios, provide significantly erroneous and unreliable results.

### SUMMARY

Aspects and advantages of embodiments of the present disclosure will be set forth in part in the following description, or can be learned from the description, or can be learned through practice of the embodiments.

One example aspect of the present disclosure is directed to a computing system that performs out-of-distribution detection. The computing system includes one or more processors and one or more non-transitory computer-readable media. The one or more non-transitory computer-readable media collectively store a machine-learned generative semantic model trained on a set of in-distribution training data (that is, the training data are samples from the distribution) comprising a plurality of in-distribution training examples. The machine-learned generative semantic model is configured to receive and process a data input to generate a first likelihood value for the data input. The first likelihood value is a first indication of the likelihood that the data input is a sample from the distribution. The one or more non-transitory computer-readable media collectively store a machine-learned generative background model trained on a set of background training data comprising a plurality of background training examples. One or more background training examples of the plurality of background training examples have been generated through perturbation of one or more in-distribution training examples of the plurality of in-distribution training examples. The machine-learned generative background model is configured to receive and process the data input to generate a second likelihood value for the data input. The second likelihood value is a second indication of the likelihood that the data input is a sample from a background distribution. The one or more non-transitory computer-readable media collectively store instructions that, when executed by the one or more processors, cause the computing system to perform operations comprising: determining a likelihood ratio value for the data input based at least in part on the first likelihood value generated by the machine-learned generative semantic model and the second likelihood value generated by the machine-learned generative background model; and predicting whether the data input is out-of-distribution based at least in part on the likelihood ratio value.

Another example aspect of the present disclosure is directed to a computer-implemented method to perform out-of-distribution detection. The method includes obtaining, by one or more computing devices, a set of in-distribution training data comprising a plurality of in-distribution training examples. The method includes training, by the one or more computing devices, a machine-learned generative semantic model using the set of in-distribution training data. The method includes perturbing, by the one or more computing devices, one or more in-distribution training examples of the plurality of in-distribution training examples to generate one or more background training examples. The method includes training, by the one or more computing devices, a machine-learned generative background model using a set of background training data that comprises the one or more background training examples. The method includes inputting, by the one or more computing devices, a data input into the machine-learned generative semantic model that has been trained on the set of in-distribution training data. The method includes receiving, by the one or more computing devices, a first likelihood value for the data input as an output of the machine-learned generative semantic model. The method includes inputting, by the one or more computing devices, the data input into the machine-learned generative background model that has been trained on the set of background training data. The method includes receiving, by the one or more computing devices, a second likelihood value for the data input as an output of the machine-learned generative background model. The method includes determining, by the one or more computing devices, a likelihood ratio value for the data input based at least in part on the first likelihood value generated by the machine-learned generative semantic model and the second likelihood value generated by the machine-learned generative background model. The method includes predicting whether the data input is out-of-distribution based at least in part on the likelihood ratio value.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system according to example embodiments of the present disclosure.

FIG. 1B depicts a block diagram of an example computing device according to example embodiments of the present disclosure.

FIG. 1C depicts a block diagram of an example computing device according to example embodiments of the present disclosure.

FIG. 2 depicts a block diagram of an example OOD detection and classification system according to example embodiments of the present disclosure.

FIG. 3 depicts a flow chart diagram of an example method to generate an OOD detection system according to example embodiments of the present disclosure.

Reference numerals that are repeated across plural figures are intended to identify the same features in various implementations.

### DETAILED DESCRIPTION

### Overview

Generally, the present disclosure is directed to systems and method to perform improved detection of out-of-distribution (OOD) inputs. In particular, current deep generative model-based approaches for OOD detection are significantly negatively affected by and struggle to distinguish population level background statistics from semantic content relevant to the in-distribution examples (that is, to distinguish between portions of in-distribution inputs which are relevant for or specific to classifying the input into in a corresponding one of a plurality of classes (“sematic content”), and portions of the in-distribution inputs which are not relevant for or less specific to classification into such classes and which may be in common with OOD inputs (“background statistics”)). In fact, such approaches have even been experimentally observed to assign higher likelihood to OOD inputs, which is opposite to the desired behavior. To resolve this problem, the present disclosure proposes a likelihood ratio method for deep generative models which effectively corrects for these confounding background statistics.

Specifically, the proposed likelihood ratio method can leverage both a semantic model that learns the semantic features of the in-distribution training examples and a background model that learns to correct for the background statistics of the training examples. In particular, the semantic model can be trained on a set of in-distribution training examples while the background model can be trained on a set of background training examples, where at least some of the background training examples are generated through corruption of the semantic content of the in-distribution training examples (e.g., through perturbation of the in-distribution training example).

After training, OOD detection for a given data input can be performed based on a likelihood ratio value generated from the respective likelihood values output by the semantic model and the background model for the data input. Use of the background model in this fashion can enhance the in-distribution specific features for OOD detection, leading to state-of-the-art performance of OOD detection.

The proposed approach has been experimentally shown to significantly outperform the raw likelihood on OOD detection for deep generative models on image datasets. As examples, U.S. Provisional Patent Application No. 62/857,774, and Jie Ren et al, “Likelihood Ratios for Out-of-distribution Detection”, arXiv: 1906.02845, which are fully incorporated by reference into and form a part of this disclosure, contain example experimental results which demonstrate the efficacy of the proposed approach in improving OOD detection.

The systems and methods of the present disclosure provide a number of technical effects and benefits. As one example, the proposed technique effectively corrects for the background components, thereby significantly improving the accuracy of OOD detection on multiple input data modalities. By improving OOD detection, the underlying accuracy of the system can be improved, for example, by preventing OOD inputs from being mis-classified by a downstream classifier. Thus, the proposed technique can improve the accuracy of a system that performs classification of inputs, leading to more improved system performance.

As one example, in a system that performs bacterial identification or other types of medical diagnosis, the number of inaccurate diagnoses (e.g., false positives) can be reduced, thereby improving the efficacy of medical care supplied based on the provided diagnoses and reducing costs associated with unnecessary and/or incorrect medical treatments. Thus, improved healthcare outcomes can be achieved at a reduced cost.

As another example technical effect and benefit, by improving OOD detection, the deployment of machine learning systems can be extended to new uses and scenarios in which classification accuracy is critical. Thus, by reducing mis-classification of OOD inputs, AI safety can be improved, allowing the benefits of machine learning systems to be brought to bear upon new problem domains or existing challenges. Furthermore, public confidence in machine learning applications can be increased.

As another example technical effect and benefit, by using the improved OOD detection systems of the present disclosure to screen inputs prior to processing by downstream system components (e.g., a classifier), the unnecessary and undesirable processing of OOD inputs by such downstream components can be reduced. Thus, computing resources such as processor usage, memory usages, network bandwidth, etc., can be conserved. Stated differently, by identifying and screening OOD inputs at an earlier stage of the processing pipeline, the wasteful application of downstream resources to such OOD inputs can be reduced.

Example implementations of the techniques described herein will now be discussed in greater detail.

### Example Notation and Problem Statement

Suppose there exists an in-distribution dataset [symbol] of (x, y) pairs sampled from the distribution p*(x, y), where x is the extracted feature vector or raw input and y∈ [symbol] :={1, . . . k, . . . , K} is the label assigning membership to one of K in-distribution classes. For simplicity, this discussion assumes inputs to be discrete, e.g., x_{d} ∈{A, C, G, T} for genomic sequences and x_{d} ∈{0, . . . ,255} for images, where d is an integer, and x_{d} denotes the d-th component of x.

In general, OOD inputs are samples (x, y) generated from an underlying distribution other than p*(x, y). As used herein, an input (x, y) is considered to be OOD if y ∈ [symbol] : that is, the class y does not belong to one of the K in-distribution classes. One goal of an OOD detection system is to accurately detect if an input x is OOD or not.

### Failure of Existing Generative Model Approaches

Certain existing methods involve computing statistics using the predictions of (ensembles of) discriminative classifiers trained on in-distribution data, e.g., taking the confidence or entropy of the predictive distribution p(y|x).

An alternative is to use generative model-based methods, which are appealing as they do not require labeled data and directly model the input distribution. These methods fit a generative model p(x) to the input data, and then evaluate the likelihood of new inputs under that model (i.e. the likelihood that a new input is drawn from p(x)). However, recent work has highlighted significant issues with this approach for OOD detection on images, showing that deep generative models such as Glow and PixelCNN sometimes assign higher likelihoods to OOD than in-distribution inputs.

Similarly, example experiments contained in the U.S. Provisional Patent Application No. 62/857,774, and Jie Ren et al, “Likelihood Ratios for Out-of-distribution Detection”, arXiv: 1906.02845, demonstrate that density estimation-based methods exhibit similar failures for OOD detection in genomics. In particular, these example experiments showed that the log-likelihood under the model is heavily affected by a sequence's GC-content. GC-content is defined as the percentage of bases that are either G or C, and is used widely in genomic studies as a basic statistic for describing overall genomic composition, and studies have shown that bacteria have an astonishing diversity of genomic GC-content, from 16.5% to 75%.

Bacteria from similar groups tend to have similar GC-content at the population level, but they also have characteristic biological patterns that can distinguish them well from each other. The confounding effect of GC-content makes the likelihood less reliable as a score for OOD detection, because an OOD input may result in a higher likelihood than an in-distribution input, because it has high GC-content and not necessarily because it contains characteristic patterns specific to the in-distribution bacterial classes.

Thus, more generally, it may be said that the failure of existing generative model approaches is attributable to the inability of the generative model to learn to distinguish background content in the new input to be analyzed from semantic content included in the in-distribution examples.

### Example Likelihood Ratio for OOD Detection

This section first provides a high level conceptual overview and then describes examples of how to adapt it to deep generative models.

Example High Level Conceptual Overview

Assume that an input x is composed of two components, (1) a background component characterized by population level background statistics, and (2) a semantic component characterized by patterns specific to the in-distribution data. For example, images can be modeled as backgrounds plus objects; text can be considered as a combination of high frequency stop words plus semantic words; genomes can be modeled as background sequences plus motifs; and/or other modalities of data can be bifurcated into semantic and background content.

More formally, for a D-dimensional input x=x₁, . . . , x_{D}, aspects of the present disclosure assume that there exists an unobserved variable z=z₁, . . . , z_{D}, where z_{d} ∈{B, S} indicates if the dth dimension of the input x_{d} is generated from the Background component or the Semantic component. Grouping the semantic and background parts, the input can be factored as x={x_{B}, x_{S}} where x_{B}={x_{d}|z_{d}=B, d=D}. For simplicity, assume that the background and semantic components are generated independently. The likelihood can be then decomposed as follows,

p(x)=p(x_{B})p(xₛ) (1)

Existing approaches to training and evaluating deep generative models do not distinguish between these two terms in the likelihood. However, the present disclosure recognizes that it may be preferred to use just the semantic likelihood p(x_{S}) to avoid the likelihood term being dominated by the background term (e.g., such that the likelihood is similar if the input is an OOD input with the same background but different semantic component). In practice, only x is observed, and it is not always easy to split an input into background and semantic parts {x_{B}, x_{S}}.

Thus, as a practical alternative, the present disclosure proposes training a background model by perturbing inputs. Adding the right amount of perturbations to inputs can corrupt the semantic structure in the data, and hence the model trained on perturbed inputs captures only the population level background statistics.

More particularly, assume that p_{θ}(·) is a model trained using in-distribution data, and p_{θ}_{₀}(·) is a background model that captures general background statistics. The present disclosure provides a likelihood ratio statistic that is defined as

$$LLR ( x ) = log \frac{{p}_{θ} ( x )}{{p}_{{θ}_{0}} ( x )} = log \frac{{p}_{θ} ( {x}_{B} ) {p}_{θ} ( {x}_{S} )}{{p}_{{θ}_{0}} ( {x}_{B} ) {p}_{{θ}_{0}} ( {x}_{S} )} , \qquad ( 2 )$$

where the factorization from Equation 1 is used.

Assume that (i) both models capture the background information equally well, that is p_{θ}(x_{B})≈p_{θ}_{₀}(x_{B}) and (ii) p_{θ}(x_{S}) is more peaky (e.g., predicts larger and more frequent likelihoods) than p_{θ}_{₀}(x_{S}) as the former is trained on data containing semantic information, while the latter model θ₀ is trained using data with noise perturbations. Then, the likelihood ratio can be approximated as

LLR(x)≈log p_{θ}(xₛ)−log p_{θ}_{₀}(x_{S}). (3)

Thus, by taking the ratio, the likelihood for the background component x_{B} is cancelled out, and only the likelihood for the semantic component x_{S} remains. As such, the proposed method produces a background contrastive score that captures the significance of the semantics compared with the background model.

Example Application of Likelihood Ratio to Auto-Regressive Models

Auto-regressive models are one of the popular choices for generating images and sequence data such as genomics, drug molecules, audio, and text. In auto-regressive models, the log-likelihood of an input can be expressed as log p_{θ}(x) =Σ_{d=1}^{D} log p_{θ}(x_{d}|x_{<d}), where x_{<d}=x₁ . . . x_{d-1}. Decomposing the log-likelihood into background and semantic parts, we have

$$log {p}_{θ} ( x ) = {\sum }_{d : {x}_{d} ∈ {x}_{B}} log {p}_{θ} ( {x}_{d} \vert {x}_{< d} ) + {\sum }_{d : {x}_{d} ∈ {x}_{S}} log {p}_{θ} ( {x}_{d} \vert {x}_{< d} ) . \qquad ( 4 )$$

A similar auto-regressive decomposition can be used for the background model p_{θ}_{₀}(x) as well. Assuming that both the models capture the background information equally well,

Σ_{d:x}_{_{d}}_{∈x}_{_{B}} log p_{θ}(x_{d}|_{<d})≈Σ_{d:x}_{_{d}}_{∈x}_{_{B}} log p_{θ}_{₀}(x_{d}|x_{<d}),

the likelihood ratio is approximated as

$$L LR ( x ) \approx {\sum }_{d : {x}_{d} ∈ {x}_{S}} log {p}_{θ} ( {x}_{d} \vert {x}_{< d} ) - {\sum }_{d : {x}_{d} ∈ {x}_{S}} log {p}_{{θ}_{0}} ( {x}_{d} \vert {x}_{< d} ) = {\sum }_{d : {x}_{d} ∈ {x}_{S}} log \frac{{p}_{θ} ( {x}_{d} \vert {x}_{< d} )}{{p}_{{θ}_{0}} ( {x}_{d} \vert {x}_{< d} )} . \qquad ( 5 )$$

Example Techniques to Train the Background Model

Any number of different techniques can be performed to perturb the in-distribution training examples to corrupt their semantic data, thereby generating background training examples.

As one example technique, noise can be added to the training example to perturb it. As one specific example, if the training example is discrete (e.g., x_{d} ∈ [symbol] ) (e.g., [symbol] ={A, C, G, T} for genomic sequences, [symbol] ={0, . . . ,255} for images, [symbol] =a dictionary of words, graphemes, phonemes, and/or n-grams for textual content, [symbol] =a set of possible amplitudes and/or frequencies for audio content, etc.), noise can be added by performing the follow operations:

| | |
|---|---|
| 1: | Generate a D-dimensional vector v = ν₁ ..., ν_{D}, where ν_{d} ∈ {0,1} are independent and |

identically distributed according to a Bernoulli distribution with rate μ.

| | |
|---|---|
| 2: | For index d ∈ {1, ..., D} do |
| 3: | If ν_{d} = 1 then |
| 4: | Sample \tilde{x}_{d} from the set [symbol] with equal probability. |
| 5: | Else |
| 6: | Set \tilde{x}_{d} = x_{d}. |
| 7: | End if |
| 8: | End for |

Thus, as one example technique, in implementations in which the training examples include a sequence of DNA characters, a computing system can add perturbations to the input data by randomly selecting positions in x₁ . . . x_{D} following an independent and identical Bernoulli distribution with rate μ and substituting the original character with one of the other characters with equal probability. The procedure is inspired by genetic mutations.

The rate μ is a hyperparameter and can be easily tuned using a small amount of a validation OOD dataset (e.g., which is different from the actual OOD dataset of interest). In the case where a validation OOD dataset is not available, μ can also be tuned using simulated OOD data. In practice, μ∈[0.1,0.2] has been shown to achieve good performance empirically.

As another example technique, semantic content included in the input (e.g., an input image) can be explicitly recognized (e.g., using a semantic segmentation model) and destroyed (e.g., removed from the image and replaced with noise or simply cropped out).

In addition or alternatively to perturbations of the input data, other techniques can improve model generalization and prevent model memorization. As one example, adding L₂ regularization with coefficient λ to model weights can help to train a good background model. In fact, adding noise to the input is equivalent to adding L₂ regularization to the model weights under some conditions. As another example, performing early stopping of the training of the background model (e.g., without or without the perturbation and/or additional regularization terms) can result in a background model that effectively cancels background statistics. Besides the methods above, adding other types of noise or regularization methods would show a similar effect.

### Example Devices and Systems

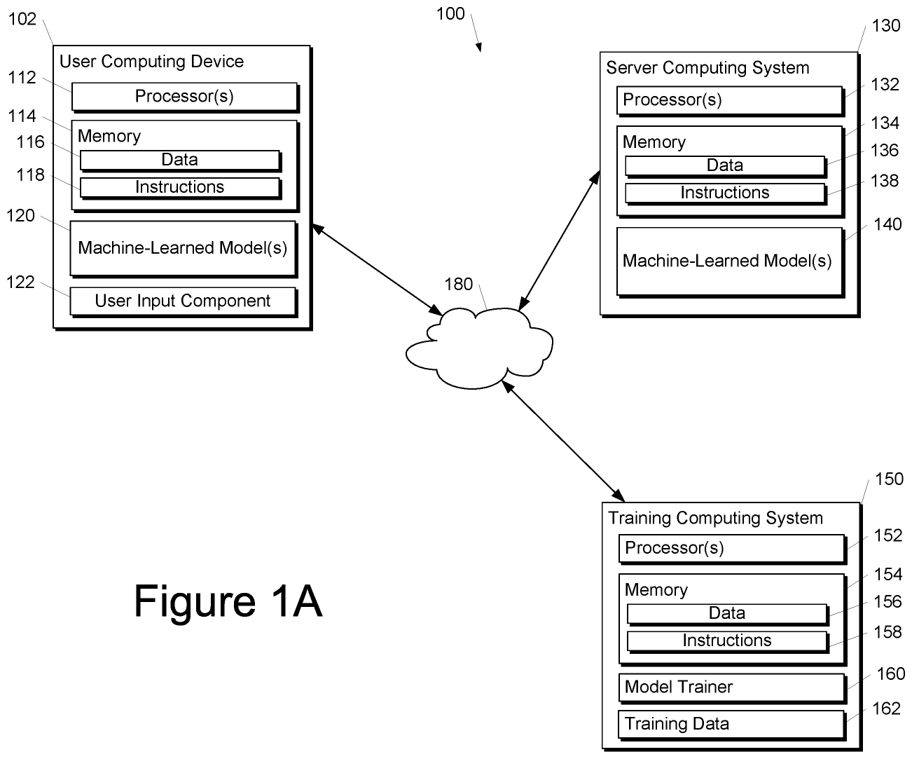

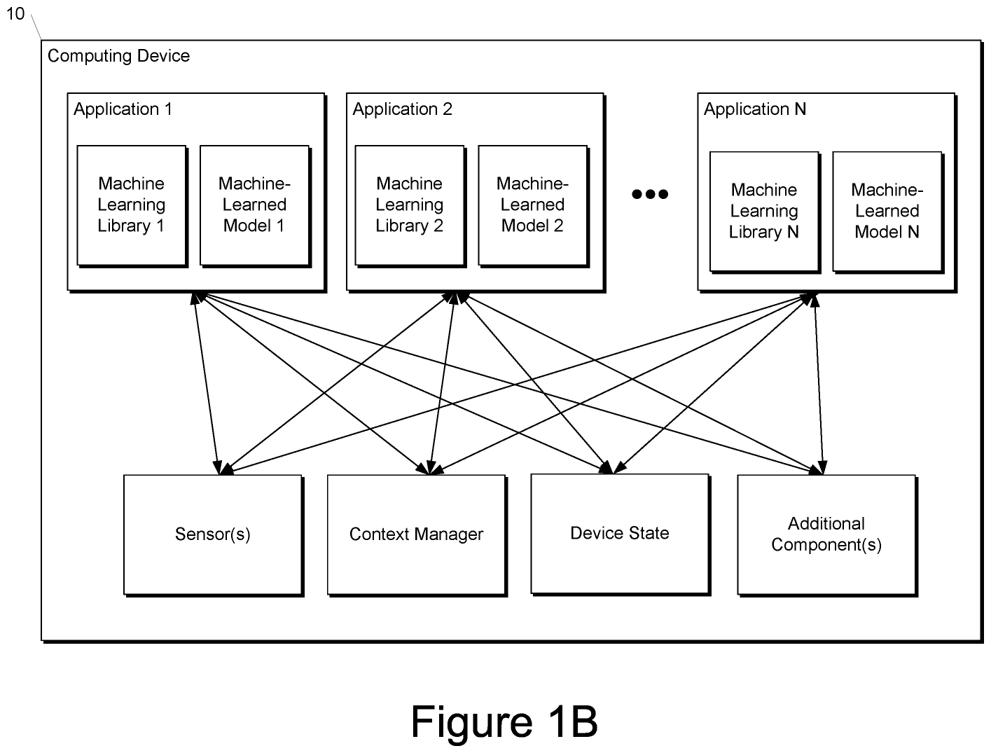

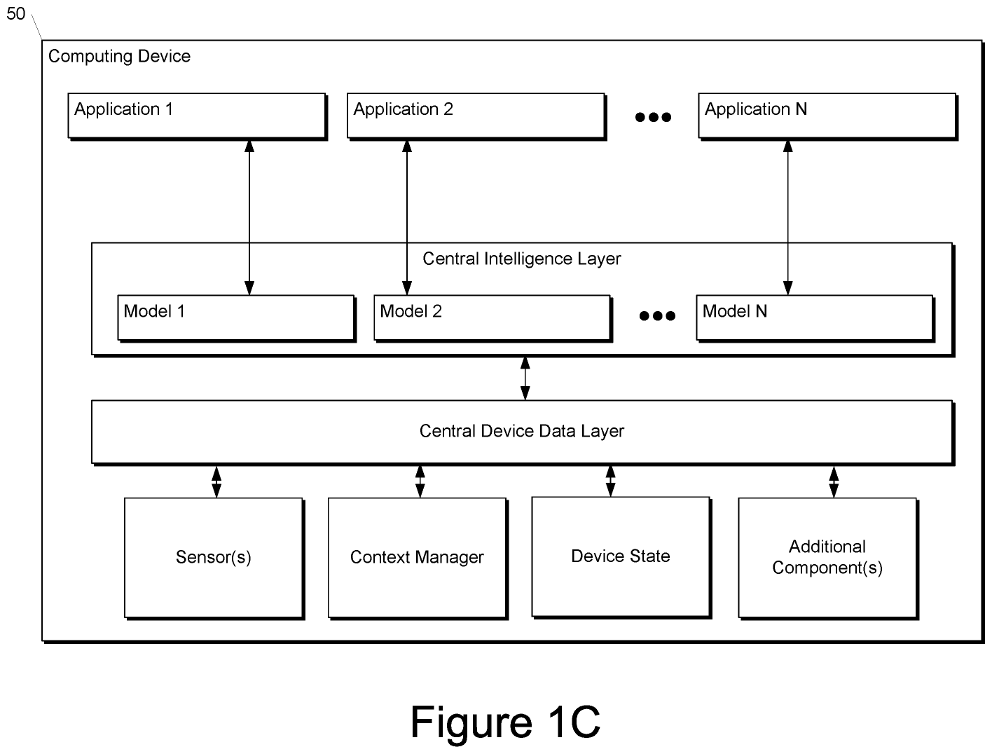

FIG. 1A depicts a block diagram of an example computing system 100 that performs OOD detection according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

In some implementations, the user computing device 102 can store or include one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks.

In some implementations, the one or more machine-learned models 120 can be received from the server computing system 130 over network 180, stored in the user computing device memory 114, and then used or otherwise implemented by the one or more processors 112. In some implementations, the user computing device 102 can implement multiple parallel instances of a single machine-learned model 120 (e.g., to perform parallel OOD detection across multiple instances of data inputs). In some implementations, the processors 112 can implement the instructions 118 to determine a likelihood ratio value for a data input based on outputs from the model(s) 120 and detect whether the data input is OOD based on the likelihood ratio value.

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service (e.g., an OOD detection service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks.

In some implementations, the processors 132 can implement the instructions 138 to determine a likelihood ratio value for a data input based on outputs from the model(s) 140 and detect whether the data input is OOD based on the likelihood ratio value.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. The training data 162 can include, for example, image data, textual data, audio data, genomic data, sensor data, etc.

In some implementations, the model trainer 160 can perform some or all of the data perturbation techniques described herein.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Model Arrangements

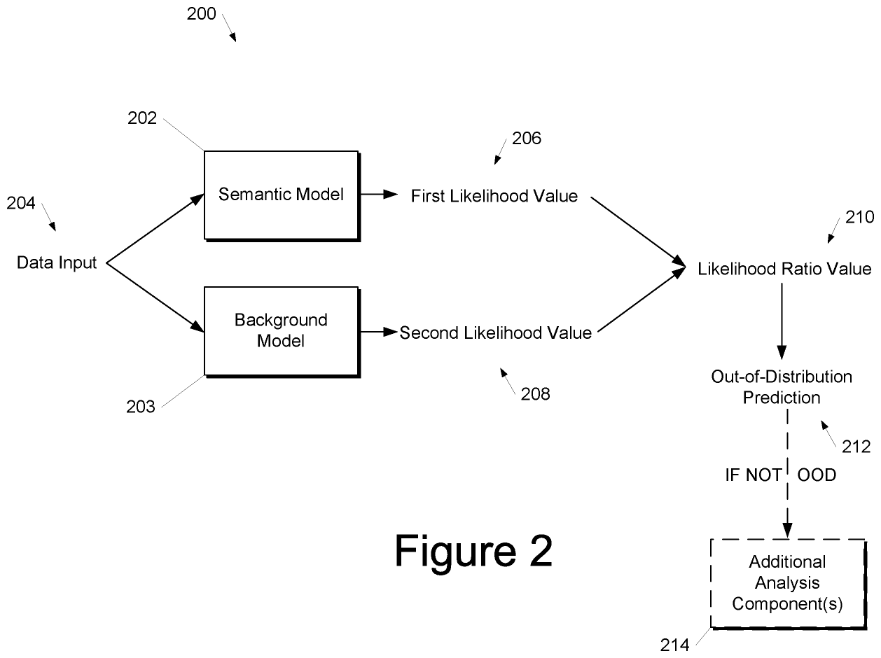

FIG. 2 depicts a block diagram of an OOD detection system 200 according to example embodiments of the present disclosure. The detection system can include a semantic model 202 and a background model 203.

The semantic model 202 can have been trained on a set of in-distribution training data that includes a plurality of in-distribution training examples. The semantic model 202 can be configured to receive and process a data input 204 to generate a first likelihood value 206 for the data input 204.

The background model 203 can have been trained on a set of background training data comprising a plurality of background training examples. In some implementations, one or more background training examples of the plurality of background training examples can have been generated through perturbation of one or more in-distribution training examples of the plurality of in-distribution training examples. The background model 203 can be configured to receive and process the data input 204 to generate a second likelihood value 208 for the data input 204.

In some implementations, one or both of the semantic model 202 and the background model 203 can be a generative model. In some implementations, one or both of the semantic model 202 and the background model 203 can be an autoregressive model. In some implementations, one or both of the semantic model 202 and the background model 203 can be a neural network such as a recurrent neural network and/or a convolutional neural network.

The OOD detection system 200 (e.g., as implemented by one or more computing devices) can determine a likelihood ratio value 210 for the data input 204 based at least in part on the first likelihood value 206 generated by semantic model 202 and the second likelihood value 208 generated by the background model 203. The system 200 can generate, based at least in part on the likelihood ratio value 210, an OOD prediction 212 that indicates whether the data input 204 is out-of-distribution.

In some implementations, determining the likelihood ratio value 210 for the data input 204 can include determining a logarithm of the first likelihood value 206 divided by the second likelihood value 208. In some implementations, generating the prediction 212 based at least in part on the likelihood ratio value 210 can include comparing the likelihood ratio value to a threshold value and predicting that the data input 204 is OOD when the likelihood ratio value 210 is less than the threshold value. The threshold value can be a hyperparameter that is user-specified or learned by the system.

In some implementations, when it is predicted that the data input 204 is not OOD, the system 200 can provide the data input 204 to one or more additional analysis components 214 such as, for example, a machine-learned classifier model for classification relative to a plurality of in-distribution classes.

### Example Methods

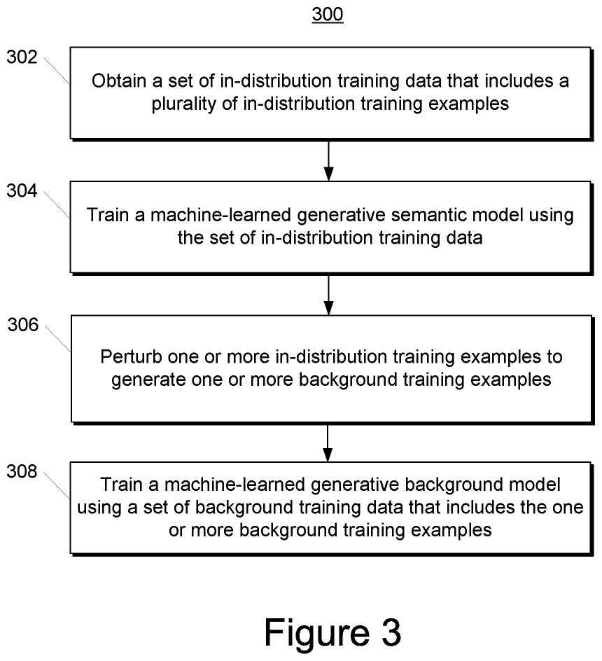

FIG. 3 depicts a flow chart diagram of an example method to perform according to example embodiments of the present disclosure. Although FIG. 3 depicts steps performed in a particular order for purposes of illustration and discussion, the methods of the present disclosure are not limited to the particularly illustrated order or arrangement. The various steps of the method 300 can be omitted, rearranged, combined, and/or adapted in various ways without deviating from the scope of the present disclosure.

At 302, a computing system can obtain a set of in-distribution training data that includes a plurality of in-distribution training examples. As examples, the in-distribution training examples can be images, genomic data, audio data, textual data, sensor data, and/or the like.

At 304, the computing system can train a machine-learned generative semantic model using the set of in-distribution training data.

At 306, the computing system can perturb one or more in-distribution training examples of the plurality of in-distribution training examples to generate one or more background training examples.

In some implementations, the perturbation of the one or more in-distribution training examples to generate the one or more background training examples can include adding noise to the one or more in-distribution training example.

In some implementations, each of the plurality of in-distribution training examples can include semantic content related to at least one of a plurality of in-distribution classes associated with the set of in-distribution training data; and the perturbation of the one or more in-distribution training examples to generate the one or more background training examples can include corrupting the semantic content included in the one or more in-distribution training examples.

In some implementations, each of the one or more in-distribution training examples can include a respective genomic sequence of DNA characters and the perturbation of the one or more in-distribution training examples to generate the one or more background training examples can include randomly mutating one or more characters of each respective genomic sequence of DNA characters to one or more alternative DNA characters.

In some implementations, each of the one or more in-distribution training examples comprises a respective image comprising a plurality of pixels and the perturbation of the one or more in-distribution training examples to generate the one or more background training examples can include, for each respective image, randomly changing respective pixel values for one or more of the plurality of pixels.

At 308, the computing system can train a machine-learned generative background model using a set of background training data that that includes the one or more background training examples.

In some implementations, the machine-learned generative semantic model can be trained at 304 using a first loss function and the machine-learned generative background model can be trained at 308 using a second loss function that is equal to the first loss function plus an additional L2 regularization term.

In some implementations, the machine-learned generative semantic model can be trained at 304 for a first number of training iterations and the machine-learned generative background model can be trained at 308 for a second number of iterations that is less than the first number of iterations. For example, the second number of iterations can be less than 50%, 60%, 70%, 80% or 90% of the first number of training iterations.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computing system that performs out-of-distribution detection, the computing system comprising:
one or more processors; and
one or more non-transitory computer-readable media that collectively store:
a machine-learned generative semantic model trained on a set of in-distribution training data comprising a plurality of in-distribution training examples which are samples of a distribution, the machine-learned generative semantic model configured to receive and process a data input to generate a first likelihood value for the data input, the first likelihood value being a first indication of the likelihood that the data input is a sample from the distribution;
a machine-learned generative background model trained on a set of background training data comprising a plurality of background training examples, one or more background training examples of the plurality of background training examples generated through perturbation of one or more in-distribution training examples of the plurality of in-distribution training examples, the machine-learned generative background model configured to receive and process the data input to generate a second likelihood value for the data input, the second likelihood value being a second indication of the likelihood that the data input is a sample from a background distribution; and
instructions that, when executed by the one or more processors, cause the computing system to perform operations comprising:
determining a likelihood ratio value for the data input based at least in part on the first likelihood value generated by the machine-learned generative semantic model and the second likelihood value generated by the machine-learned generative background model; and
predicting whether the data input is out-of-distribution based at least in part on the likelihood ratio value.

2. The computing system of claim 1, wherein determining the likelihood ratio value for the data input comprises determining a logarithm of the first likelihood value divided by the second likelihood value.

3. The computing system of claim 1, wherein predicting whether the data input is out-of-distribution based at least in part on the likelihood ratio value comprises:
comparing the likelihood ratio value to a threshold value; and
predicting that the data input is out-of-distribution when the likelihood ratio value is less than the threshold value.

4. The computing system of claim 1, wherein the operations further comprise:
when it is predicted that the data input is not out-of-distribution, providing the data input to a machine-learned classifier model for classification relative to a plurality of in-distribution classes.

5. The computing system of claim 1, wherein the perturbation of the one or more in-distribution training examples to generate the one or more background training examples comprises adding noise to the one or more in-distribution training examples.

6. The computing system of claim 1, wherein the data input comprises a genomic sequence.

7. The computing system of claim 1, wherein each of the one or more in-distribution training examples comprises a respective genomic sequence of DNA characters and the perturbation of the one or more in-distribution training examples to generate the one or more background training examples comprises randomly mutating one or more characters of each respective genomic sequence of DNA characters to one or more alternative DNA characters.

8. The computing system of claim 1, wherein one or both of the machine-learned generative semantic model and the machine-learned generative background model comprise a recurrent neural network or a convolutional neural network.

9. The computing system of claim 1, wherein the data input comprises an image.

10. The computing system of claim 1, wherein each of the one or more in-distribution training examples comprises a respective image comprising a plurality of pixels and the perturbation of the one or more in-distribution training examples to generate the one or more background training examples comprises, for each respective image, randomly changing respective pixel values for one or more of the plurality of pixels.

11. The computing system of claim 1, wherein:
each of the plurality of in-distribution training examples comprises semantic content related to at least one of a plurality of in-distribution classes associated with the set of in-distribution training data; and
the perturbation of the one or more in-distribution training examples to generate the one or more background training examples corrupts the semantic content included in the one or more in-distribution training examples.

12. (canceled)

13. (canceled)

14. (canceled)

15. A computer-implemented method to perform out-of-distribution detection, the method comprising:
obtaining, by one or more computing devices, a set of in-distribution training data comprising a plurality of in-distribution training examples;
training, by the one or more computing devices, a machine-learned generative semantic model using the set of in-distribution training data;
perturbing, by the one or more computing devices, one or more in-distribution training examples of the plurality of in-distribution training examples to generate one or more background training examples;
training, by the one or more computing devices, a machine-learned generative background model using a set of background training data that comprises the one or more background training examples;
inputting, by the one or more computing devices, a data input into the machine-learned generative semantic model that has been trained on the set of in-distribution training data;
receiving, by the one or more computing devices, a first likelihood value for the data input as an output of the machine-learned generative semantic model;
inputting, by the one or more computing devices, the data input into the machine-learned generative background model that has been trained on the set of background training data;
receiving, by the one or more computing devices, a second likelihood value for the data input as an output of the machine-learned generative background model;
determining, by the one or more computing devices, a likelihood ratio value for the data input based at least in part on the first likelihood value generated by the machine-learned generative semantic model and the second likelihood value generated by the machine-learned generative background model; and
predicting whether the data input is out-of-distribution based at least in part on the likelihood ratio value.

16. The computer-implemented method of claim 15, wherein determining, by the one or more computing devices, the likelihood ratio value for the data input comprises determining, by the one or more computing devices, a logarithm of the first likelihood value divided by the second likelihood value.

17. The computer-implemented method of claim 15, wherein predicting, by the one or more computing devices, whether the data input is out-of-distribution based at least in part on the likelihood ratio value comprises:
comparing, by the one or more computing devices, the likelihood ratio value to a threshold value; and
predicting, by the one or more computing devices, that the data input is out-of-distribution when the likelihood ratio value is less than the threshold value.

18. The computer-implemented method of claim 15, further comprising:
when it is predicted that the data input is not out-of-distribution, providing, by the one or more computing devices, the data input to a machine-learned classifier model for classification relative to a plurality of in-distribution classes.

19. The computer-implemented method of claim 15, wherein perturbing, by the one or more computing devices, the one or more in-distribution training examples of the plurality of in-distribution training examples to generate the one or more background training examples comprises adding, by the one or more computing devices, noise to each of the one or more in-distribution training examples.

20. The computer-implemented method of claim 15, wherein the data input comprises a genomic sequence.

21. The computer-implemented method of claim 15, wherein each of the one or more in-distribution training examples comprises a respective genomic sequence of DNA characters and perturbing, by the one or more computing devices, the one or more in-distribution training examples of the plurality of in-distribution training examples to generate the one or more background training examples comprises randomly mutating, by the computing device for each of the one or more in-distribution training examples, one or more characters of the respective genomic sequence of DNA characters to alternative DNA characters.

22. (canceled)

23. (canceled)

24. The computer-implemented method of claim 15, wherein each of the one or more in-distribution training examples comprises a respective image that comprises a plurality of pixels and perturbing, by the one or more computing devices, the one or more in-distribution training examples of the plurality of in-distribution training examples to generate the one or more background training examples comprises randomly changing, by the computing device for each of the one or more in-distribution training examples, one or more pixel values of the respective image to an alternative values.

25. The computer-implemented method of claim 15, wherein:
each of the plurality of in-distribution training examples comprises respective semantic content related to at least one of a plurality of in-distribution classes associated with the set of in-distribution training data; and
perturbing, by the one or more computing devices, the one or more in-distribution training examples of the plurality of in-distribution training examples to generate the one or more background training examples comprises corrupting, by the one or more computing devices, the respective semantic content included in each of the one or more in-distribution training examples.

26. (canceled)

27. The computer-implemented method of claim 15, wherein:
training, by the one or more computing devices, the machine-learned generative semantic model comprises training, by the one or more computing devices, the machine-learned generative semantic model using a first loss function; and
training, by the one or more computing devices, the machine-learned generative background model comprises training, by the one or more computing devices, the machine-learned generative background model using a second loss function that comprises the first loss function with an additional L2 regularization term added.

28. The computer-implemented method of claim 15, wherein:
training, by the one or more computing devices, the machine-learned generative semantic model comprises training, by the one or more computing devices, the machine-learned generative semantic model for a first number of training iterations; and
training, by the one or more computing devices, the machine-learned generative background model comprises training, by the one or more computing devices, the machine-learned generative background model for a second number of training iterations that is less than the first number of training iterations.

29. The computer-implemented method of claim 15, wherein one or both of the machine-learned generative semantic model and the machine-learned generative background model comprise an auto-regressive model.

30. (canceled)
